(19) United States
(12) Patent Application Publication
(10) Pub. No.: US 2004/0096820 A1
(43) Pub. Date: May 20, 2004

(54) Comparative proteomics of progressor and nonprogressor populations

(75) Inventors: William E. Rich, Redwood Shores, CA (US); David D. Ho, Chappaqua, NY (US); Linqi Zhang, Rochelle Park, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Ciphergen Biosystems, Inc., Fremont, CA; Aaron Diamond AIDS Research Center, New York, NY; The Rockefeller University, New York, NY

(21) Appl. No.: 10/452,666

(22) Filed: May 30, 2003

Related U.S. Application Data
(63) Continuation-in-part of application No. 10/452,763, filed on May 30, 2003.
(60) Provisional application No. 60/412,397, filed on Sep. 21, 2002.
(60) Provisional application No. 60/412,414, filed on Sep. 20, 2002.
(60) Provisional application No. 60/405,595, filed on Aug. 23, 2002.
(60) Provisional application No. 60/384,428, filed on May 31, 2002.
(60) Provisional application No. 60/412,414, filed on Sep. 20, 2002.
(60) Provisional application No. 60/405,595, filed on Aug. 23, 2002.
(60) Provisional application No. 60/384,428, filed on May 31, 2002.

(51) Int. Cl. C12Q 1/70; G01N 33/53; G01N 33/574; G01N 33/554; G01N 33/569

(52) U.S. Cl. 435/5; 435/7.32; 435/7.23; 435/7.1

(57) ABSTRACT

The invention identifies polypeptide biomarkers of disease progression or nonprogression by comparative protein profiling of samples from progressors and nonprogressors subpopulations of a population exposed to the pathogen or sharing a risk facto causing the disease. The polypeptides, their ligands, and modulators find use as diagnostic, prognostic, and therapeutic agents.

### BACKGROUND OF THE INVENTION

It is well known that different persons, both exposed to the same pathogenic agent, can respond to the exposure in different ways. It would be useful to discover one or more biomarkers that could distinguish the two classes of individuals, particularly in a prognostic, diagnostic, or therapeutics context. For example, some persons infected with HIV develop AIDS, while other persons infected with HIV do not develop AIDS. Such persons are referred to as “long term nonprogressors.”

This invention addresses this need and others.

### BRIEF SUMMARY OF THE INVENTION

This invention provides methods for discovering polypeptide (e.g., protein and peptide) biomarkers that differentiate progressor and nonprogressor subpopulations within a population which has been exposed to a known pathogenic agent or has a known common risk factor for a disease. The method involves comparing polypeptide or protein profiles from samples from at least two such subpopulations, and identifying one or more polypeptides that serve as a biomarker, or biomarker pattern, that helps to distinguish among such subpopulations.

In another aspect, the invention provides methods of diagnosis, prognosis, or assessing the susceptibility to disease progression based upon the detection or quantitation of such an identified biomarker or pattern in a subject before, upon, or subsequent to an exposure to the pathogen or acquisition of the risk factor. In another aspect, the invention provides methods of treatment wherein the polypeptide biomarker(s) or a modulator of any biological activity thereof is administered to a nonprogressor so as to reduce the likelihood or severity of such progression.

In a first aspect, therefore, the invention provides a method for identifying polypeptide markers of disease progression or nonprogression in a population by identifying a population having a common risk factor for the disease or exposure to a pathological agent known to cause the disease, classifying the population into disease progressor and nonprogressor subpopulations, obtaining biological samples from members of the two subpopulations, collectively or individually protein profiling the samples, and comparing the sample protein profiles for the two subpopulations so as to identify polypeptide biomarkers whose expression differs between the two such populations.

In some embodiments, the levels of the biomarkers between two such classes, differ by at least 25%, 50%, 100%, 2-fold, 4-fold, or 10-fold. In some embodiments, the statistical description of the biomarker distribution in the two classes, results in an individual to be assigned to one class or another with a false positive rate of less than 20%, 10%, or 5% (e.g., less than 20%, 10%, or 5% of a member of the nonprogressor class being assigned to the progressor class) based upon the individual and classes' protein profile or polypeptide biomarker(s) levels. In some embodiments, the statistical description of the biomarker distribution in the two classes, typically results in a member of the progressor class to be assigned to the nonprogressor class with a false negative rate of less than 20%, 10%, or 5%.

In some embodiments, the progressor and nonprogressor populations have been exposed to an infectious pathogenic agent known to cause the disease. In some embodiments, the infectious agent is selected from the group consisting of plant and animal parasites, bacteria, fungi, mold, yeast, viruses and prions. In one such embodiment, the virus is a retrovirus. In other embodiments, the virus is HIV, HCV, CMV, or HBV, or a viral agent causing encephalitis.

In some embodiments, the progressor and nonprogressor populations have been exposed to a non-infectious environmental agent known to be a cause of the disease. In some embodiments such an agent is a known toxic chemical or known toxic drug (e.g., smoke and other combustion products, industrial chemicals, pesticides, cosmetics, food additive). In some embodiments, such a non-infectious agent agent is a non-chemical agent with a known adverse health effect such as radiation. Examples of radiation include electromagnetic radiation such as X-rays, gamma-rays, radio waves, microwaves, UV light, visible light and infrared light (e.g., sunlight).

In some embodiments, the risk factor is a characteristic shared by the progressor and nonprogressor populations which risk factor is known to be associated with an increased likelihood of the disease. Such risk factors can include non-environmental and environmental risk factors. In some embodiments, the non-environmental factors may, for instance, be familial (e.g., a family history of a disease), genetic (e.g., possession of a particular gene known to be associated with a disease); cultural (e.g., a diet or cultural practice or membership known to be associated with a particular disease), occupational, age-related or health status-related factors known to be associated with the particular disease.

In some embodiments, the protein profiling involves performing a proteomic analysis on a direct or indirect sample from a member or members of both progressor and nonprogressor populations.

In some embodiments, the polypeptides to be profiled are from 500 to 5000 daltons or 1,000 to 10,000 daltons. In some embodiments, the profile includes a comparison of at least 1, 10, 20, 50, 100, 200, 400, 1,000, or up to 5,000 polypeptides in a single detection scheme. In some embodiments, multiple detection schemes may be used to increase the number of the proteins profiled.

In one embodiment, the protein profiling method comprises SELDI mass spectrometry of the samples.

In another embodiment, differences in protein expression between the progressors and nonprogressors are detected using pattern recognition software.

In another aspect of the invention, a differentially expressed protein once identified can then be used to identify a binding partner or a modulator of the biological activity of the protein. In one embodiment, for example, the protein can be immobilized on a solid phase. Then, candidate proteins are contacted with the immobilized protein. Proteins that bind with the immobilized protein are detected by any of a number of ways including for example, fluorescence detection (if the candidates are labeled) or mass spectrometry (e.g., SELDI).

In another aspect of the invention, the binding partner may be useful as a probe in diagnostic and prognostic testing or as a therapeutic agent.

In another aspect of the invention, the polypeptide biomarker may be used as a therapeutic agent.

In one of its aspects, the invention therefore provides methods for identifying candidate modulators of such a polypeptide by a) docking to a solid support a polypeptide that is differentially expressed between a progressor population and a nonprogressor population; b) contacting the docked polypeptide with at least one candidate ligand for the protein; and c) detecting binding between the docked polypeptide and at least one candidate ligand. In a further embodiment, the binding is detected by SELDI or immunoassay.

In one aspect, the invention provides a method comprising the steps of a) profiling proteins in a sample from at least one member of a first population exposed to a pathogenic agent wherein the pathogenic agent evokes a particular pathophysiological response in the first population, whereby the first population is defined as a progressor population; profiling proteins in a sample from at least one member of a second population exposed to the pathogenic agent wherein the pathogenic agent does not evoke the pathophysiological response in the second population, whereby the second population is defined as a nonprogressor population; and c) detecting differentially expressed proteins between the first and second samples. In one embodiment, the at least one samples are a plurality of samples, each sample from a different individual. In another embodiment, the agent is a drug or drug candidate and the pathophysiological response is a known drug toxicity. In other embodments, the pathogenic agent is an infectious agent such as a bacterium, a virus or a prion. In other embodiments, the agent is a toxic chemical or cancer causing agent.

In additional embodiments, the above profiling is performed using a method selected from the group consisting of MADLI, SELDI, two-dimensional gel electrophoresis, protein array analysis, population two-hybrid screening, and multiplexed immunoassay. In some embodiments, the protein or polypeptide profiling method does not comprise a biospecific absorbion step. In some embodiments, the profiling profiling method is based upon the physico-chemical properties of the polypeptides and not their biological activities or ligand or antibody affinities.

In some embodiments, the known pathologic agent to which the progressor and nonprogressor population has been exposed is not a carcinogen, mutagen or is not a chemical agent or is not a drug. In other embodiments, the disease of progression is not cancer or a form of cancer. In other embodiments, the known pathologic agent is not infectious, or is not a virus, or not a bacterium, or not a prion. In other embodiments, the known agent is not a physical agent such as radiation. In other embodiments, the identified polypeptide biomarker is differentially expressed in exposure niave or risk factor free subjects and can be used to indicate susceptibility or not to progression in the event of exposure or acquisition of a risk factor. In such embodiments, the identified polypeptide biomarker distinguishes or helps to idetify individuals as progressors or nonprogressors in the absence of any exposure for the known pathogen or any risk for the known risk factor of the individual. In some embodiments, the polypeptide marker to be identified was not an unrecognized or unknown biomarker for said progression or nonprogression.

### BRIEF DESCRIPTION OF THE DRAWINGS

FIG. 1 is a schematic illustration of the use of proteomic profiling to identify differences in the pattern of protein expression between progressor and nonprogressor populations and the subsequent use of the differently expressed proteins as prognostic indicators or as molecular targets or probes in the development of therapeutic agents.

### DETAILED DESCRIPTION OF THE INVENTION

A. Definitions

It is noted here that as used in this specification and the appended claims, the singular forms “a,” “an,” and “the” include plural reference unless the context clearly dictates otherwise.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Biological sample” refers to a sample derived from a virus, cell, tissue, organ or organism (either eukaryotic or prokaryotic) including, without limitation, cell, tissue or organ lysates or homogenates, or body fluid samples, such as blood, urine, sputum, or cerebrospinal fluid. Such samples include, but are not limited to, tissue isolated from humans, or explants, primary, and transformed cell cultures derived therefrom. Biological samples may also include sections of tissues such as frozen sections taken for histologic purposes. A biological sample can be obtained from a procaryotic organism or a eukaryotic organism such as fungi, plants, insects, protozoa, birds, fish, reptiles, and preferably a mammal such as rat, mice, cow, dog, guinea pig, or rabbit, and most preferably a primate such as chimpanzees or humans.

“Biopolymer” refers to a polymer of biological origin, e.g., polypeptides, polynucleotides, polysaccharides or polyglycerides (e.g., di- or tri-glycerides).

“Polypeptide” refers to a polymer composed of amino acid residues and related naturally occurring structural variants (e.g., glycoproteins, phosphoproteins, lipoproteins) thereof linked via peptide bonds. The term “protein” typically refers to large polypeptides. The term “peptide” typically refers to short polypeptides. Polypeptides may have molecular weights of less than 10,000 daltons, 5,000 daltons, or 2,000 daltons. In some embodiments, the polypeptide has a molecular weight of from about 500 to about 10,000 daltons, more preferably between about 500 to about 5,000 daltons, or 500 to 3,000 daltons.

“Detectable moiety” or a “label” refers to a composition detectable by spectroscopic, photochemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which antisera or monoclonal antibodies are available. The detectable moiety often generates a measurable signal, such as a radioactive, chromogenic, or fluorescent signal, that can be used to quantitate the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavadin. The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavidin. The binding partner may itself be directly detectable, for example, an antibody may be itself labeled with a fluorescent molecule. Quantitation of the signal can be achieved by the strength of the measured signal from a labeled moiety., e.g., scintillation counting, densitometry, or flow cytometry.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from open reading frames that flank the gene and encode proteins other than protein encoded by the gene. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Purify” or “purification” means removing at least one contaminant from the composition to be purified. Purification does not require that the purified compound be 100% pure.

“Plurality” means at least two.

A “ligand” is a compound that specifically binds to a target molecule (e.g., a receptor).

A “receptor” is compound that specifically binds to a ligand.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. This term also encompasses, e.g., polyclonal, monoclonal, single-chain, humanized, chimeric antibodies, and fragments thereof.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A ligand or a receptor (e.g., an antibody) “specifically binds to” or “is specifically immunoreactive with” a compound analyte when the ligand or receptor functions in a binding reaction which is determinative of the presence of the analyte in a sample of heterogeneous compounds. Thus, under designated assay (e.g., immunoassay) conditions, the ligand or receptor binds preferentially to a particular analyte and does not bind in a significant amount to other compounds present in the sample. For example, a polynucleotide specifically binds under hybridization conditions to an analyte polynucleotide comprising a complementary sequence; an antibody specifically binds under immunoassay conditions to an antigen analyte bearing an epitope against which the antibody was raised; and an adsorbent specifically binds to an analyte under proper elution conditions.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter which can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of mass spectrometry to detect gas phase ions.

“Ion source” refers to a sub-assembly of a gas phase ion spectrometer that provides gas phase ions. In one embodiment, the ion source provides ions through a desorption/ionization process. Such embodiments generally comprise a probe interface that positionally engages probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm² to 50 mJ/mm². Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device that can be used to introduce ions derived from an analyte into a gas phase ion spectrometer, such as a mass spectrometer. A “probe” will generally comprise a solid substrate (either flexible or rigid) comprising a sample presenting surface on which an analyte is presented to the source of ionizing energy. “SELDI probe” refers to a probe comprising an adsorbent (also called a “capture reagent”) attached to the surface. “Adsorbent surface” refers to a surface to which an adsorbent is bound. “Chemically selective surface” refers to a surface to which is bound either an adsorbent or a reactive moiety that is capable of binding a capture reagent, e.g., through a reaction forming a covalent or coordinate covalent bond.

“Mass analyzer” refers to a subassembly of a mass spectrometer that comprises means for measuring a parameter which can be translated into mass-to-charge ratios of gas phase ions. In a time-of flight mass spectrometer the mass analyzer comprises an ion optic assembly, a flight tube and an ion detector.

“Fluence” refers to the energy delivered per unit area of interrogated image.

“Tandem mass spectrometer” refers to any mass spectrometer that is capable of performing two successive stages of m/z-based discrimination or measurement of ions, including of ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that are capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Laser desorption mass spectrometer” refers to a mass spectrometer which uses laser as a means to desorb, volatilize, and ionize an analyte.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip)

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Surface-Enhanced Neat Desorption” or “SEND” is a version of SELDI that involves the use of probes comprising energy absorbing molecules chemically bound to the probe surface. (“SEND probe.”)

“Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(trimethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Energy absorbing molecules” (“EAM”) refer to molecules that are capable of absorbing energy from a laser desorption ionization source and thereafter contributing to the desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid. It also includes EAMs used in SELDI.

“Adsorbent” or “capture reagent” refers to any material capable of binding an analyte (e.g., a target polypeptide). “Chromatographic adsorbent” refers to a material typically used in chromatography. Chromatographic adsorbents include, for example, ion exchange materials, metal chelators, hydrophobic interaction adsorbents, hydrophilic interaction adsorbents, dyes, mixed mode adsorbents (e.g., hydrophobic attraction/electrostatic repulsion adsorbents). “Biospecific adsorbent” refers an adsorbent comprising a biomolecule, e.g., a nucleotide, a nucleic acid molecule, an amino acid, a polypeptide, a simple sugar, a polysaccharide, a fatty acid, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than a chromatographic adsorbent. Further examples of adsorbents for use in SELDI can be found in U.S. Pat. No. 6,225,047 (Hutchens and Yip, “Use of retentate chromatography to generate difference maps,” May 1, 2001).

“Reactive moiety” refers to a chemical moiety that is capable of binding a capture reagent. Epoxide and carbodiimidizole are useful reactive moieties to covalently bind polypeptide capture reagents. Nitrilotriacetic acid is a useful reactive moiety to bind metal chelating agents through coordinate covalent bonds.

“Adsorption” refers to detectable noncovalent binding of an analyte to an adsorbent or capture reagent.

“Analyte” refers to any component of a sample that is desired to be detected. The term can refer to a single component or a plurality of components in the sample.

“Monitoring” refers to recording changes in a continuously varying parameter.

The “complexity” of a sample adsorbed to an adsorption surface of an affinity capture probe means the number of different protein species that are adsorbed.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Monitoring” refers to recording changes in a continuously varying parameter.

“Solid support” refers to a solid material which can be derivatized with, or otherwise attached to, a chemical moiety, such as a capture reagent, a reactive moiety or an energy absorbing species. Exemplary solid supports include chips (e.g., probes), microtiter plates and chromatographic resins.

“Chip” refers to a solid support having a generally planar surface to which a chemical moiety may be attached. Chips that are adapted to engage a probe interface are also called “probes.”

“Molecular binding partners” and “specific binding partners” refer to pairs of molecules, typically pairs of biomolecules, which exhibit specific binding. Molecular binding partners include, without limitation, receptor and ligand, antibody and antigen, biotin and avidin, and biotin and streptavidin.

“Biochip” refers to a chip to which a chemical moiety is attached. Frequently, the surface of the biochip comprises a plurality of addressable locations, each of which location has the chemical moiety attached there.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, Q-10, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, Q-10, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu⁺⁺ and Ni⁺⁺, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US 2003 0032043 A1 (Pohl and Papanu, “Latex Based Adsorbent Chip,” Jul. 16, 2002) and U.S. patent application 60/350,110 (Um et al., “Hydrophobic Surface Chip,” Nov. 8, 2001); U.S. patent application 60/367,837, (Boschetti et al., “Biochips With Surfaces Coated With Polysaccharide-Based Hydrogels,” May 5, 2002) and U.S. patent application entitled “Photocrosslinked Hydrogel Surface Coatings” (Huang et al., filed Feb. 21, 2003).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

B. General Description

In one aspect, the invention provides a method for identifying biomarkers of disease progression or nonprogression in a population by identifying a population having a common risk factor for the disease or exposure to a pathological agent known to cause the disease, classifying the population into disease progressor and disease nonprogressor classes, obtaining biological samples from members of the two classes, collectively or individually protein profiling the samples, and comparing the sample protein profiles for the two classes so as to identify polypeptides whose expression differ between the two classes.

In another aspect, the invention provides a method for identifying differentially expressed polypeptides which can be used as receptors, targets or probes for drug development by identifying a population having a common risk factor for the disease or exposure to a pathological agent known to cause the disease, classifying the population into disease progressor and disease nonprogressor classes, obtaining biological samples from members of the two classes, collectively or individually protein profiling the samples, and comparing the sample protein profiles for the two classes so as to identify polypeptides whose expression differ between the two classes.

The population on which such methods is practiced can be any group of living organisms. In general, members of both the progressor and nonprogressor subpopulations will be members of a larger population exposed to the known pathogenic agent or sharing a known common risk factor. However, in some members of the group (the first subpopulation), the exposure or risk factor will evoke a pathophysiological response of some kind. These members are referred to herein as “progressors.” Members in whom the exposure or risk factor does not result in a pathophysiological response (the second subpopulation) are referred to herein as “nonprogressors.” The number of individuals in each subpopulation can be at least 1, at least 10, at least 100 or at least 1000. Progressor and nonprogressor subpopulations may be distinguished or defined by the time course or severity of their progression or both. For instance, these subpopulations may be differentiated by the rate at which they progress to the pathophysiological state or they may be differentiated by the severity of the disease they develop. Progressor and nonprogressor subpopulations may be further stratified and matched for comparison according to the degree of their exposure or the magnitude of the risk factors. In some embodiments, the progressor and nonprogressor subpopulations are mammalian (e.g., human, mouse, rat). The preferred population is human. In some embodiments, the population is an animal population or a plant population.

The pathophysiological response can be any response indicating pathophysiology. The response can occur at the organism, organ system, organ, tissue, cellular or biochemical level. The pathophysiological response can be a disease state (e.g., AIDS, diabetes, asthma, depression, schizophrenia, obesity, atherosclerosis, hepatitis, neurodegenerative illness) or a symptom of disease (e.g., high blood pressure, low CD4+ cell count, fatigue).

The method involves performing a proteomic analysis on a sample from a member or members of both progressor and nonprogressor subpopulations. The sample can be any biological sample from individuals, or a derivative thereof. For example, the sample can be a direct sample, such as a blood, urine, cerebrospinal fluid, or tissue sample. Alternatively, the sample can be an indirect sample, for example, cells from the individuals can be cultured and the culture supernatant can be the sample to be profiled.

Proteomic analysis involves protein profiling of the sample. “Protein profiling” as used herein means the detection of a plurality of different proteins in the sample. The plurality of proteins preferably is at least 10 proteins, at least 25 proteins, at least 100 proteins or at least 500 proteins in the sample. Protein profiling can include pretreatment of the samples. For example, samples can be pre-fractionated to simplify the proteins profiled. Alternatively, proteins can be fragmented before analysis. One version of fragmentation before analysis is ICAT, a method in mass spectrometry (See International PCT Publication No. WO 00/11208 (Aebersold et al., “Rapid quantitative analysis of proteins or protein function in complex mixtures,” Mar. 2, 2000).

In some embodiments, the polypeptides to be profiled are from 500 to 5,000 daltons or 1,000 to 10,000 daltons.

Several methods of protein profiling are known in the art. They include, for example, protein biochip analysis, chromatography and gel electrophoresis (e.g., 2D gel electrophoresis). Protein biochip analysis can involve the use of high throughput biochips, in which a single addressable location detects many proteins, or multi-point detection biochips, in which a single addressable location captures a single or a few proteins. Chromatographic SELDI biochips are examples of high throughput chips. Protein arrays in which different, specific capture reagents are located at individual addressable locations are examples of multipoint protein biochips. The many methods of detection on a biochip are described herein. However, mass spectrometry, and in particular SELDI mass spectrometry, is particularly useful because of its high-throughput capability. Samples can be profiled on a single kind of surface or on a plurality of different surfaces. A plurality of different surfaces is preferable at least in the initial screening because it increases the opportunity of detecting proteins that are differentially expressed.

The result of a protein profile is an indication of the presence or absence of a plurality of different proteins in each sample or, preferably, a quantitative measurement of the amounts of each of the plurality of proteins in each sample.

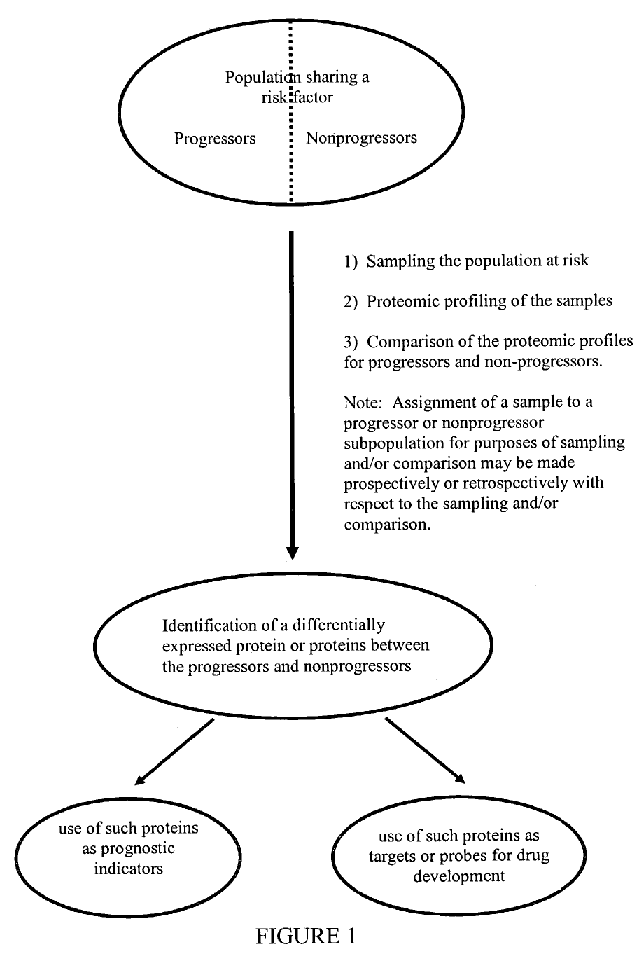

After samples from progressors and nonprogressors are profiled, the profiles are compared or analyzed to detect qualitative and/or quantitative differences in protein expression or patterns of protein expression. In one embodiment, detecting differences in expression involves comparing the expression of the proteins detected in a sample from a progressor with the expression of proteins detected in a sample from a nonprogressor. Relative amounts of a protein between the two samples is a preferable comparison to presence versus absence of a protein between the two samples. In this way, the investigator can detect proteins that can function as diagnostic or prognostic markers or as drug targets. FIG. 1 is an illustration of one embodiment of such an approach.

In another embodiment, differences in protein expression between the progressors and nonprogressors are detected using pattern recognition software. Pattern recognition software includes an algorithm that produces a classifier using selected elements of the protein profiles. The classifier can then be used to classify an unknown sample as coming from a progressor or a nonprogressor. Such software is described, for example, in International PCT Patent Publication No. WO 02/42733 (Paulse et al., “Method for Analyzing Mass Spectra,” May 30, 2002) and International PCT Patent Publication No. WO 02/06829 (Hitt et al., “A processor for discriminating between biological states based on hidden patterns from biological data,” Jan. 24, 2002). Such patterns are useful in diagnostic and prognostic tests to determine whether an individual is a progressor or nonprogressor. Proteins that are useful in classifying a subject sample as a progressor on nonprogressor are referred to as “biomarkers.” In a typical diagnostic test, a sample from a test subject is analyzed to detect one or more biomarkers or biomarker pattern characteristic of progressors or nonprogressors. The existence or amount of the biomarkers or pattern in the sample is compared with a standard or classifying pattern to classify the sample into one group or another.

Differentially expressed proteins may then be further characterized. For example, a differentially expressed protein can identified. A first step generally involves fractionating the sample to obtain a relatively pure protein. Many methods are known for identifying proteins. In one method, the protein is cleaved into fragments, e.g., with a protease having a known cleavage site, the mass of the fragments is determined, e.g., by mass spectrometry, and a protein database is searched to identify candidate proteins that would produce peptides of the detected masses upon cleavage. Another methodology involves tandem mass spectrometry. In this method, a protein is fragmented using, e.g., a proteolytic enzyme. One of the resulting peptides is selected for further analysis by the first mass spectrometer. The fragment undergoes collisional cooling, resulting in a peptide ladder. The peptide ladder is analyzed in the second mass spectrometer and the amino acid sequence of the fragment is determined by protein ladder sequencing. The amino acid sequences of one or more fragments of the peptides are used to query a protein database to identify identity candidates for the protein. See, e.g., International PCT Patent Publication No. WO 02/31491 (Weinberger et al., “Apparatus and methods for affinity capture tandem mass spectrometry,” Apr. 18, 2002).

A differentially expressed protein can then be used to identify a binding partner. For example, the protein can be immobilized on a solid phase. Then, candidate proteins are contacted with the immobilized protein. Proteins that bind with the immobilized protein are detected by any of a number of ways including for example, fluorescence detection (if the candidates are labeled) or mass spectrometry (e.g., SELDI).

Once identified, antibodies against the differentially expressed proteins can be produced by any of the known means, including commercially if the protein is already known and such antibodies are commercially available. Such antibodies are now useful, for example, in immuno-detection assays to detect the protein. Such detection methods may be useful in diagnostic and prognostic testing.

A differentially expressed protein may be useful as a drug if, for example, it is found in greater quantities in nonprogressor populations. The protein can be formulated in a pharmaceutical composition for administration to a person at risk of progressing to a disease state. Where the polypeptide biomarker is associated with progression, antibodies directed to such biomarkers may have therapeutic as well as diagnostic and prognostic utility as could be understood by one of ordinary skill in the art.

The three dimensional structure of the identified, differentially expressed protein can be determined by, for example, X-ray crystallography. Structural information can be used to identify the active site of the protein and the structure of small molecules that can bind to the active site.

The interaction between a differentially expressed protein and its binding partner may involve the mechanism that allows either progression or nonprogression to disease. Accordingly, either protein may be a target for pharmaceutical intervention, i.e., a drug target. Compounds, such as small organic molecules, can be screened, either individually or in libraries, to determine whether they affect or modulate the binding between the differentially expressed protein and its binding partner or substrate. Screening methods are well known in the art. Typically, one member of the binding pair is immobilized on a solid support. The immobilized protein is contacted with its binding partner and the test molecule or molecules. The effect on binding is determined compared with binding outside the presence of the test molecule. Test molecules that affect binding are drug candidates for further testing.

Modulators of the activity of a biomarker may be administered as a method of treatment so as to prevent progression. In one of its aspects, the invention therefore provides methods for identifying candidate modulators of such a biomarker or polypeptide by a) docking to a solid support a polypeptide that is differentially expressed between a progressor population and a nonprogressor population; b) contacting the docked polypeptide with at least one candidate ligand for the protein; and c) detecting binding between the docked polypeptide and at least one candidate ligand. In a futher embodiment, the binding is detected by SELDI or immunoassay.

C. Methods

As described herein, each of these techniques can be used, alone or in combination, to identify a candidate polypeptide (e.g., protein or peptide, or fragment thereof) or set of candidate polypeptides of interest that are differentially expressed in a progressor and a nonprogressor population or samples therefrom. Potential polypeptides of interest include, e.g., ion channels, receptors (e.g., G protein coupled receptors) cytokines, chemokines, signal transduction proteins, housekeeping proteins, cell cycle regulation proteins, transcription factors, zinc finger proteins, chromatin remodeling proteins, membrane associated polypeptides, HLA-antigens, glycoppolypeptides, hormones, enzymes, antigenic peptides and proteins, intracellular polypeptides, extracellular fluid polypeptides. Polypeptides found in bodily fluids such as urine, cerebrospinal fluid, blood, and plasma are also of particular interest.

Using the protein analysis tools described below, one or more of the physio-chemical characteristics of the protein can be used fractionate the proteins of interest, while reducing background and increasing sensitivity of protein detection. In this manner, a polypeptide expression profile of a nonprogressor and progressor can be compared to each other to identify polypeptides which are differentially expressed in progressor and nonprogressors exposed to the same agent or sharing the same risk factor. This information can be used to diagnostically distinguish such progressors and nonprogressors. The information can also be used to develop of pharmaceutical therapies which administer such polypeptides or modulators of their functions or amounts to a subject so as to modulate a disease or health state associated with the nonprogressor or progressor status.

Protein Fractionation Analysis of Samples

Polypeptides in the sample can be fractionated based on at least one physio-chemical property of the polypeptide. Such means are known to one of ordinary skill in the art. Molecular mass, isoelectric points, hydrophilicity or hydrophobicity, metal chelate binding ability are properties which can be used to fractionate polypeptides. Amino acid sequence also can indicate whether the polypeptide includes glycosylation or phosphorylation sites. Post-translational modifications of the polypeptide will be reflected in changes to molecular weight. Epitopes, in turn, may be targets for antibody binding.

A most useful method of separation is molecular weight, as there are many useful methods to separate proteins based on this characteristic including, for example, SDS gel electrophoresis and gas phase ion spectrometry, e.g., mass spectrometry. Another useful physiochemical characteristic is isoelectric point. Isoelectric focusing, affinity chromatography and solid phase extraction on an ion exchange resin will fractionate proteins in a sample based on this property.

Methods of fractionating proteins can be used to determine the amount of polypeptide in a sample. The use of one or more elected physiochemical characteristics can enhance the sensitivity of fractionation and reduce background. The techniques described herein can be used to examine one or more proteins expressed in a cell, up to tens, hundreds, thousands, or tens of thousands of proteins. Any one technique or a combination of techniques can be used to fractionate the proteins, based on one or more physio-chemical property. Methods of fractionation include, e.g., two dimensional gels; capillary gel electrophoresis; mass spectrometry, e.g., MALDI, SELDI; ICAT (isotope coded affinity tag, see, e.g., Mann, Nature Biotechnology 17:954-955 (1999); Gygi et al., Nature Biotechnology 17:994-999 (1999)); chromatography, e.g., gel-filtration, ion-exchange, affinity, immunoaffinity, and metal chelate chromatography, HPLC, e.g., reversed phase, ion-exchange, and size exclusion HPLC; western blotting; immunohistochemistry techniques such as ELISA and in situ screening with antibodies, etc (see, e.g., Blackstock & Weir, Trends in Biotech. 17:121-127 (1999); Dutt & Lee, Biochemical Engineering, pages 176-179 (April 2000); Page et al., Drug Discovery Today 4:55-62 (1999); Wang & Hewick, Drug Discovery Today 4:129-133 (1999); Regnier et al., Trends in Biotech. 17:101-106 (1999); and Pandey & Mann, Nature 405:837-846 (2000)).

In one embodiment, two-dimensional electrophoresis can be used to fractionate the proteins of the invention. This technique fractionates proteins based on the physio-chemical characteristics of pI and molecular weight. 2d gel electrophoresis and the techniques described herein can be used alone, or in combination with other techniques such as mass spectrometry, e.g., MALDI and SELDI, described herein below.

In another embodiment, described below, MALDI is a mass spectrometry technique that fractionates proteins based on mass, and is often combined with size and or affinity chromatography techniques to increase resolution.

In another embodiment, described below, SELDI is a mass spectrometry technique that couples affinity fractionation with mass spectrometry. An affinity matrix or probe based on such polypeptide properties as, pI (ion exchange resin and wash), antibody binding, glycosylation, phosphorylation, histidine residues used in SELDI, in combination with mass spectrometry, to identify proteins with high resolution, accuracy, and sensitivity. When using this technique, an affinity matrix that enriches for the candidate polypeptides can be determined, based on the physio-chemical characteristics of the protein encoded by the transcript.

Mass Spectrometry Analysis of Samples

Polypeptides or fragments thereof can be analyzed using mass spectrometry methods. This method fractionates the polypeptides based on mass. In certain embodiments laser-desorption/ionization mass spectrometry is used to analyze the sample on the substrate-bound adsorbent.

Modern laser desorption/ionization mass spectrometry (“LDI-MS”) can be practiced in several main variations: Liquid chromatography-mass spectrometry (LC-MS), matrix assisted laser desorption/ionization (“MALDI”) mass spectrometry and surface-enhanced laser desorption/ionization (“SELDI”). Mass spectrometers can be further coupled to a quadrupole time-of-flight mass spectrometer. In LC-MS, fractions from a liquid chromatograph are introducted by electrospray into a mass spectrometer. In MALDI, the analyte, which may contain biological molecules, is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with affinity reagents that selectively bind the analyte. In another variant, the surface is derivatized with energy absorbing molecules that are not desorbed when struck with the laser. In another variant, the surface is derivatized with molecules that bind the analyte and that contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens and Yip, “Method and apparatus for desorption and ionization of analytes). The two methods can be combined by, for example, using a SELDI affinity surface to capture an analyte and adding matrix-containing liquid to the captured analyte to provide the energy absorbing material.

In certain embodiments, the laser desorption/ionization mass spectrophotometer is further coupled to a quadrupole time-of-flight mass spectrometer QqTOF MS (see, e.g., Weinberger et al., WO 02/31491 and Krutchinsky et al., WO 99/38185). Methods such as MALDI-QqTOFMS (Krutchinsky et al., WO 99/38185; Shevchenko et al. (2000) Anal. Chem. 72: 2132-2141), ESI-QqTOF MS (Figeys et al. (1998) Rapid Comm'ns. Mass Spec. 12-1435-144) and chip capillary electrophoresis (chip-CE)-QqTOF MS (Li et al. (2000) Anal. Chem. 72: 599-609) have been described previously.

Retentate Chromatography

Retentate chromatography is a method for the multidimensional resolution of analytes in a sample. The method involves (1) selectively adsorbing analytes from a sample to a substrate under a plurality of different adsorbent/eluant combinations (“selectivity conditions”) and (2) detecting the retention of adsorbed analytes by desorption spectrometry. Each selectivity condition provides a first dimension of separation, separating adsorbed analytes from those that are not adsorbed. Desorption mass spectrometry provides a second dimension of separation, separating adsorbed analytes from each other according to mass. Because retentate chromatography involves using a plurality of different selectivity conditions, many dimensions of separation are achieved. The relative adsorption of one or more analytes under the two selectivity conditions also can be determined. This multidimensional separation provides both resolution of the analytes and their characterization.

Further, the analytes thus separated remain docked in a retentate map that is amenable to further manipulation to examine, for example, analyte structure and/or function. Also, the docked analytes can, themselves, be used as adsorbents to dock other analytes exposed to the substrate. In sum, the present invention can provide a rapid, multidimensional and high information resolution of analytes.

The method can take several forms. In one embodiment, the analyte is adsorbed to two different adsorbents at two physically different locations and each adsorbent is washed with the same eluant (selectivity threshold modifier). In another embodiment, the analyte is adsorbed to the same adsorbent at two physically different locations and washed with two different eluants. In another embodiment, the analyte is adsorbed to two different adsorbents in physically different locations and washed with two different eluants. In another embodiment, the analyte is adsorbed to an adsorbent and washed with a first eluant, and retention is detected; then, the adsorbed analyte is washed with a second, different eluant, and subsequent retention is detected.

Methods of Performing Retentate Chromatography

Retentate chromatography is a particularly useful method for fractionating polypeptides in a sample. According to this method, the polypeptides are fractionated on a solid phase adsorbent which binds polypeptides based on particular physio-chemical properties. Unbound polypeptides are washed away. Then the retained polypeptides are further fractionated by mass spectrometry, thereby providing fractionation based on at least two physio-chemical properties.

The sample containing the analyte may be contacted to the adsorbent either before or after the adsorbent is positioned on the substrate using any suitable method which will enable binding between the analyte and the adsorbent. The adsorbent can simply be admixed or combined with the sample. The sample can be contacted to the adsorbent by bathing or soaking the substrate in the sample, or dipping the substrate in the sample, or spraying the sample onto the substrate, by washing the sample over the substrate, or by generating the sample or analyte in contact with the adsorbent. In addition, the sample can be contacted to the adsorbent by solubilizing the sample in or admixing the sample with an eluant and contacting the solution of eluant and sample to the adsorbent using any of the foregoing techniques (i.e., bathing, soaking, dipping, spraying, or washing over).

Contacting the analyte to the adsorbent: Exposing the sample to an eluant prior to binding the analyte to the adsorbent has the effect of modifying the selectivity of the adsorbent while simultaneously contacting the sample to the adsorbent. Those components of the sample which will bind to the adsorbent and thereby be retained will include only those components which will bind the adsorbent in the presence of the particular eluant which has been combined with the sample, rather than all components which will bind to the adsorbent in the absence of elution characteristics which modify the selectivity of the adsorbent.

The sample should be contacted to the adsorbent for a period of time sufficient to allow the analyte to bind to the adsorbent. Typically, the sample is contacted with the analyte for a period of between about 30 seconds and about 12 hours. Preferably, the sample is contacted to the analyte for a period of between about 30 seconds and about 15 minutes.

The temperature at which the sample is contacted to the adsorbent is a function of the particular sample and adsorbents selected. Typically, the sample is contacted to the adsorbent under ambient temperature and pressure conditions, however, for some samples, modified temperature (typically 4° C. through 37° C.) and pressure conditions can be desirable and will be readily determinable by those skilled in the art.

Numerous different experiments can be conducted on a very small amount of sample. Generally, a volume of sample containing from a few attomoles to 100 picomoles of analyte in about 1 μl to 500 μl is sufficient for binding to the adsorbent. Analyte may be preserved for future experiments after binding to the adsorbent because any adsorbent locations which are not subjected to the steps of desorbing and detecting all of the retained analyte will retain the analyte thereon. Therefore, in the case where only a very small fraction of sample is available for analysis, the present invention provides the advantage of enabling a multitude of experiments with different adsorbents and/or eluants to be carried out at different times without wasting sample.

Washing the Adsorbent with Eluants: After the sample is contacted with the analyte, resulting in the binding of the analyte to the adsorbent, the adsorbent is washed with eluant. Typically, to provide a multi-dimensional analysis, each adsorbent location is washed with at least a first and a second different eluants. Washing with the eluants modifies the analyte population retained on a specified adsorbent. The combination of the binding characteristics of the adsorbent and the elution characteristics of the eluant provide the selectivity conditions which control the analytes retained by the adsorbent after washing. Thus, the washing step selectively removes sample components from the adsorbent.

The washing step can be carried out using a variety of techniques. For example, as seen above, the sample can be solubilized in or admixed with the first eluant prior to contacting the sample to the adsorbent. Exposing the sample to the first eluant prior to or simultaneously with contacting the sample to the adsorbent has, to a first approximation, the same net effect as binding the analyte to the adsorbent and subsequently washing the adsorbent with the first eluant. After the combined solution is contacted to the adsorbent, the adsorbent can be washed with the second or subsequent eluants.

Washing an adsorbent having the analyte bound thereto can be accomplished by bathing, soaking, or dipping the substrate having the adsorbent and analyte bound thereon in an eluant; or by rinsing, spraying, or washing over the substrate with the eluant. The introduction of eluant to small diameter spots of affinity reagent is best achieved by a microfluidics process.

When the analyte is bound to adsorbent at only one location and a plurality of different eluants are employed in the washing step, information regarding the selectivity of the adsorbent in the presence of each eluant individually may be obtained. The analyte bound to adsorbent at one location may be determined after each washing with eluant by following a repeated pattern of washing with a first eluant, desorbing and detecting retained analyte, followed by washing with a second eluant, and desorbing and detecting retained analyte. The steps of washing followed by desorbing and detecting can be sequentially repeated for a plurality of different eluants using the same adsorbent. In this manner the adsorbent with retained analyte at a single location may be reexamined with a plurality of different eluants to provide a collection of information regarding the analytes retained after each individual washing.

The foregoing method is also useful when adsorbents are provided at a plurality of predetermined addressable locations, whether the adsorbents are all the same or different. However, when the analyte is bound to either the same or different adsorbents at a plurality of locations, the washing step may alternatively be carried out using a more systematic and efficient approach involving parallel processing. Namely, the step of washing can be carried out by washing an adsorbent at a first location with eluant, then washing a second adsorbent with eluant, then desorbing and detecting the analyte retained by the first adsorbent and thereafter desorbing and detecting analyte retained by the second adsorbent. In other words, all of the adsorbents are washed with eluant and thereafter analyte retained by each is desorbed and detected for each location of adsorbent. If desired, after detection at each adsorbent location, a second stage of washings for each adsorbent location may be conducted followed by a second stage of desorption and detection. The steps of washing all adsorbent locations, followed by desorption and detection at each adsorbent location can be repeated for a plurality of different eluants. In this manner, and entire array may be utilized to efficiently determine the character of analytes in a sample. The method is useful whether all adsorbent locations are washed with the same eluant in the first washing stage or whether the plurality of adsorbents are washed with a plurality of different eluants in the first washing stage.

Detection

Analytes retained by the adsorbent after washing are adsorbed to the substrate. Analytes retained on the substrate are detected by desorption spectrometry: desorbing the analyte from the adsorbent and directly detecting the desorbed analytes.

Methods For Desorption: Desorbing the analyte from the adsorbent involves exposing the analyte to an appropriate energy source. Usually this means striking the analyte with radiant energy or energetic particles. For example, the energy can be light energy in the form of laser energy (e.g., UV laser) or energy from a flash lamp. Alternatively, the energy can be a stream of fast atoms. Heat may also be used to induce/aid desorption.

Methods of desorbing and/or ionizing analytes for direct analysis are well known in the art. One such method is called matrix-assisted laser desorption/ionization, or MALDI. In MALDI, the analyte solution is mixed with a matrix solution and the mixture is allowed to crystallize after being deposited on an inert probe surface, trapping the analyte within the crystals may enable desorption. The matrix is selected to absorb the laser energy and apparently impart it to the analyte, resulting in desorption and ionization. Generally, the matrix absorbs in the UV range. MALDI for large proteins is described in, e.g., U.S. Pat. No. 5,118,937 (Hillenkamp et al.) and U.S. Pat. No. 5,045,694 (Beavis and Chait).

Surface-enhanced laser desorption/ionization, or SELDI, represents a significant advance over MALDI in terms of specificity, selectivity and sensitivity. SELDI is described in U.S. Pat. No. 5,719,060 (Hutchens and Yip). SELDI is a solid phase method for desorption in which the analyte is presented to the energy stream on a surface that enhances analyte capture and/or desorption. In contrast, MALDI is a liquid phase method in which the analyte is mixed with a liquid material that crystallizes around the analyte.

One version of SELDI, called SEAC (Surface-Enhanced Affinity Capture), involves presenting the analyte to the desorbing energy in association with an affinity capture device (i.e., an adsorbent). It was found that when an analyte is so adsorbed, it can be presented to the desorbing energy source with a greater opportunity to achieve desorption of the target analyte. An energy absorbing material can be added to the probe to aid desorption. Then the probe is presented to the energy source for desorbing the analyte

Another version of SELDI, called SEND (Surface-Enhanced Neat Desorption), involves the use of a layer of energy absorbing material onto which the analyte is placed. A substrate surface comprises a layer of energy absorbing molecules chemically bond to the surface and/or essentially free of crystals. Analyte is then applied alone (i.e., neat) to the surface of the layer, without being substantially mixed with it. The energy absorbing molecules, as do matrix, absorb the desorbing energy and cause the analyte to be desorbed. This improvement is substantial because analytes can now be presented to the energy source in a simpler and more homogeneous manner because the performance of solution mixtures and random crystallization is eliminated. This provides more uniform and predictable results that enable automation of the process. The energy absorbing material can be classical matrix material or can be matrix material whose pH has been neutralized or brought into the basic range. The energy absorbing molecules can be bound to the probe through covalent or noncovalent means.

Another version of SELDI, called SEPAR (Surface-Enhanced Photolabile Attachment and Release), involves the use of photolabile attachment molecules. A photolabile attachment molecule is a divalent molecule having one site covalently bound to a solid phase, such a flat probe surface or another solid phase, such as a bead, that can be made part of the probe, and a second site that can be covalently bound with the affinity reagent or analyte. The photolabile attachment molecule, when bound to both the surface and the analyte, also contains a photolabile bond that can release the affinity reagent or analyte upon exposure to light. The photolabile bond can be within the attachment molecule or at the site of attachment to either the analyte (or affinity reagent) or the probe surface.

Method for Direct Detection of Analytes.

The desorbed analyte can be detected by any of several means. When the analyte is ionized in the process of desorption, such as in laser desorption/ionization ass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Preferably, the method is laser desorption ionization.

Selectivity Conditions

One advantage of the invention is the ability to expose the analytes to a variety of different binding and elution conditions, thereby providing both increased resolution of analytes and information about them in the form of a recognition profile. As in conventional chromatographic methods, the ability of the adsorbent to retain the analyte is directly related to the attraction or affinity of the analyte for the adsorbent as compared to the attraction or affinity of the analyte for the eluant or the eluant for the adsorbent. Some components of the sample may have no affinity for the adsorbent and therefore will not bind to the adsorbent when the sample is contacted to the adsorbent. Due to their inability to bind to the adsorbent, these components will be immediately separated from the analyte to be resolved. However, depending upon the nature of the sample and the particular adsorbent utilized, a number of different components can initially bind to the adsorbent.

Adsorbents

Adsorbents are the materials that bind analytes. A plurality of adsorbents can be employed in retentate chromatography. Different adsorbents can exhibit grossly different binding characteristics, somewhat different binding characteristics, or subtly different binding characteristics. Adsorbents which exhibit grossly different binding characteristics typically differ in their bases of attraction or mode of interaction. The basis of attraction is generally a function of chemical or biological molecular recognition. Bases for attraction between an adsorbent and an analyte include, for example, (1) a salt-promoted interaction, e.g., hydrophobic interactions, thiophilic interactions, and immobilized dye interactions; (2) hydrogen bonding and/or van der Waals forces interactions and charge transfer interactions, such as in the case of a hydrophilic interactions; (3) electrostatic interactions, such as an ionic charge interaction, particularly positive or negative ionic charge interactions; (4) the ability of the analyte to form coordinate covalent bonds (i.e., coordination complex formation) with a metal ion on the adsorbent; (5) enzyme-active site binding; (6) reversible covalent interactions, for example, disulfide exchange interactions; (7) glycoprotein interactions; (8) biospecific interactions; or (9) combinations of two or more of the foregoing modes of interaction. That is, the adsorbent can exhibit two or more bases of attraction, and thus be known as a “mixed functionality” adsorbent.

Eluants

The eluants, or wash solutions, selectively modify the threshold of absorption between the analyte and the adsorbent. The ability of an eluant to desorb and elute a bound analyte is a function of its elution characteristics. Different eluants can exhibit grossly different elution characteristics, somewhat different elution characteristics, or subtly different elution characteristics.

As in the case of adsorbents, eluants which exhibit grossly different elution characteristics generally differ in their basis of attraction. For example, various bases of attraction between the eluant and the analyte include charge or pH, ionic strength, water structure, concentrations of specific competitive binding reagents, surface tension, dielectric constant and combinations of two or more of the above.

Variability of Two Parameters

The ability to provide different binding characteristics by selecting different adsorbents and the ability to provide different elution characteristics by washing with different eluants permits variance of two distinct parameters each of which is capable of individually effecting the selectivity with which analytes are bound to the adsorbent. The fact that these two parameters can be varied widely assures a broad range of binding attraction and elution conditions so that the methods of the present invention can be useful for binding and thus detecting many different types of analytes.

The selection of adsorbents and eluants for use in analyzing a particular sample will depend on the nature of the sample, and the particular analyte or class of analytes to be characterized, even if the nature of the analytes are not known. Typically, it is advantageous to provide a system exhibiting a wide variety of binding characteristics and a wide variety of elution characteristics, particularly when the composition of the sample to be analyzed is unknown. By providing a system exhibiting broad ranges of selectivity characteristics, the likelihood that the analyte of interest will be retained by one or more of the adsorbents is significantly increased.

One skilled in the art of chemical or biochemical analysis is capable of determining the selectivity conditions useful for retaining a particular analyte by providing a system exhibiting a broad range of binding and elution characteristics and observing binding and elution characteristics which provide the best resolution of the analyte. Because the present invention provides for systems including broad ranges of selectivity conditions, the determination by one skilled in the art of the optimum binding and elution characteristics for a given analyte can be easily accomplished without the need for undue experimentation.

Analytes

The present invention permits the resolution of analytes based upon a variety of biological, chemical, or physio-chemical properties of the analyte by exploiting the properties of the analyte through the use of appropriate selectivity conditions. Among the many properties of analytes which can be exploited through the use of appropriate selectivity conditions are the hydrophobic index (or measure of hydrophobic residues in the analyte), the isoelectric point (i.e., the pH at which the analyte has no charge), the hydrophobic moment (or measure of amphipathicity of an analyte or the extent of asymmetry in the distribution of polar and nonpolar residues), the lateral dipole moment (or measure of asymmetry in the distribution of charge in the analyte), a molecular structure factor (accounting for the variation in surface contour of the analyte molecule such as the distribution of bulky side chains along the backbone of the molecule), secondary structure components (e.g., helix, parallel and antiparallel sheets), disulfide bands, solvent-exposed electron donor groups (e.g., His), aromaticity (or measure of pipi interaction among aromatic residues in the analyte) and the linear distance between charged atoms.

These are representative examples of the types of properties which can be exploited for the resolution of a given analyte from a sample by the selection of appropriate selectivity characteristics in the methods of the present invention. Other suitable properties of analytes which can form the basis for resolution of a particular analyte from the sample will be readily known and/or determinable by those skilled in the art and are contemplated by the instant invention.

Identification of Proteins Fractionated by Mass Spectrometry

The data of a mass spectrum can be used to identify the proteins present in a sample by executing an algorithm with a programmable digital computer that compares the MS data to records in a database. Each molecule provides characteristic mass-spectrometric (MS) data (also referred to as a mass spectral “signature” or “fingerprint”) when analyzed by MS methods. This data can be analyzed by comparing it to databases containing, inter alia, actual or theoretical MS data or biopolymer sequence information. Additionally, a molecule may be cleaved into fragments for MS analysis. Information obtained from the MS analysis of fragments is also compared to a database to identify polypeptides in the analyte (Yates, J. Mass Spec. 33: 1-19 (1988); Yates et al., U.S. Pat. No. 5,538,897; Yates et al., U.S. Pat. No. 6,017,693).

Further methods for identifying proteins detected by SELDI are described, e.g., in U.S. Pat. No. 6,225,047; International Patent Application PCT/US00/28163, and U.S. S No. 60/277,677, filed Mar. 20, 2001.

Data generated by desorption and detection of polypeptides can be analyzed using any suitable means. In one embodiment, data is analyzed with the use of a programmable digital computer. The computer program generally contains a readable medium that stores codes. Certain code can be devoted to memory that includes the location of each feature on a substrate, the identity of the adsorbent at that feature and the elution conditions used to wash the adsorbent. Using this information, the program can then identify the set of features on the substrate defining certain selectivity characteristics (e.g., types of adsorbent and eluants used). The computer also contains code that receives as input, data on the strength of the signal at various molecular masses received from a particular addressable location on the substrate. This data can indicate the number of polypeptides detected, optionally including the strength of the signal and the determined molecular mass for each polypeptide detected.

In certain embodiments, MS data and information obtained from that data are compared to a database consisting of data and information relating to biopolymers. For example, the database may consist of sequences of nucleotides or amino acids. The database may consist of nucleotide or amino acid sequences of expressed sequence tags (ESTs). Alternatively, the database may consist of sequences of genes at the nucleotide or amino acid level. The database can include, without limitation, a collection of nucleotide sequences, amino acid sequences, or translations of nucleotide sequences included in the genome of any species.

A database of information relating to biopolymers, e.g., sequences of nucleotides or amino acids, is typically analyzed via a computer program or a search algorithm which is optionally performed by a computer. Information from sequence databases is searched for best matches with data and information obtained from the methods of the present invention (see e.g., Yates (1998) J. Mass Spec. 33: 1-19; Yates et al., U.S. Pat. No. 5,538,897; Yates et al., U.S. Pat. No. 6,017,693).

Any appropriate algorithm or computer program useful for searching a database can be used. Search algorithms and databases are constantly updated, and such updated versions will be used in accordance with the present invention. Examples of programs or databases can be found on the World Wide Web (WWW) at http://base-peak.wiley.com/, http://mac-mann6.embl-heidelberg.de/MassSpec/Software.html, http://www.mann.emblheidelberg.de/Services/PeptideSearch/PeptideSearchIntro.html, ftp://ftp.ebi.ac.uk/pub/databases/, and http://donatello.ucsf.edu. U.S. Pat. Nos. 5,632,041; 5,964,860; 5,706,498; and 5,701,256 also describe algorithms or methods for sequence comparison.

In one embodiment, the database of protein, peptide, or nucleotide sequences is a combination of databases. Examples of databases include, but are not limited to, ProteinProspector at the UCSF web site (prospector.ucsf.edu), the Genpept database, the GenBank database (described in Burks et al. (1990) Methods in Enzymology 183: 3-22, EMBL data library (described in Kahn et al. (1990) Methods in Enzymology 183:23-31, the Protein Sequence Database (described in Barker et al. (1990) Methods in Enzymology 183: 31-49, SWISS-PROT (described in Bairoch et al. (1993) Nucleic Acids Res., 21: 3093-3096, and PIR-International (described in (1993) Protein Seg. Data Anal. 5:67-192).

In a further embodiment, novel databases are generated for comparison to mass spectrometrically determined MS data, e.g., mass or mass spectra of cleaved protein and peptide fragments. For example, a theoretical database of all the possible amino acid sequence combinations of the peptide masses being characterized is generated (Parekh et al., WO 98/53323). Then, the database is compared with the actual masses determined using mass spectrometry to determine the amino acid sequence of the peptides in the sample.

In some embodiments, the mass of a polypeptide derived from a mass spectrum is used to query a database for those masses of proteins or predicted proteins from nucleic acid sequences that provide the closest fit. In this manner, an unknown protein can be rapidly identified without an amino acid sequence. In other embodiments of the invention, the masses provided from chimeric polypeptide fragments thereof can be compared to the predicted mass spectra of a database of proteins or predicted proteins from a nucleic acid sequences that provide the closest fit. An algorithm or computer program generates a theoretical cleavage of sequences in a database with the same cleavage agent used to cleave the biopolymer analyzed by MS methods.

Sequences or simulated cleavage fragments from the sequence database that fall within a desired range of similar sequence homologies to sequences generated from the MS data of parent or fragment molecules are designated “matches” or “hits.” In this manner, the identity of the test domain or fragments thereof can be rapidly determined. The investigator can customize or vary the range of acceptable sequence homology comparison values according to each particular analysis.

In another embodiment, retention assays are performed under the same set of selectivity thresholds on two different cell types, and the retention data from the two assays is compared. Differences in the retention maps (e.g., presence or strength of signal at any feature) indicate analytes that are differentially expressed by the two cells. This can include, for example, generating a difference map indicating the difference in signal strength between two retention assays, thereby indicating which analytes are increasingly or decreasingly retained by the adsorbent in the two assays.

Classification and Comparison of Proteomic Profile Data or Spectra

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent. The groups may be further stratified according to degree of pathoology or effect or with respect to additional risk factors.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002 0193950 A1 (Gavin et al., “Method or analyzing mass spectra,” Dec. 19, 2002); U.S. 2003 0004402 A1 (Hitt et al., “Process for discriminating between biological states based on hidden patterns from biological data,” Jan. 2, 2003); and U.S. 2003 0055615 A1 (Zhang and Zhang, “Systems and methods for processing biological expression data” Mar. 20, 2003).

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

### EXAMPLES

The following examples are offered to illustrate, but not to limit the claimed invention.

### Example 1

A pathogenic agent is known to induce a pathophysiological response in some people, while other people are immune to the agent. A group of individuals who have been exposed to a pathogenic agent are identified. The group includes both people and who are progressors and nonprogressors. A sample, such as a blood sample, is prepared from each individual. The samples are prepared for profiling by fractionation on a chromatographic column. The fractionated samples are profiled using a SELDI biochip surface. A plurality of such biochip surfaces may be used. For instance, two different SELDI biochip surfaces, e.g., an anion exchange surface and a metal chelate surface, may be used. This involves placing each sample on a different spot on a chip, allowing binding of the proteins, washing away unbound proteins, applying a matrix to each of the spots, and detecting proteins on each spot by mass spectrometry on a Ciphergen ProteinChip® System (Ciphergen Biosystems, Fremont, Calif., USA).

The mass spectra generated by each sample are then analyzed by ProteinChip® software. ProteinChip® software identifies over 500 peaks in each mass spectrum, each peak representing a protein. The spectra are downloaded into Biomarker Patterns™ Software (Ciphergen Biosystems, Fremont, Calif., USA). Biomarker Patterns™ Software creates a spreadsheet in which each row represents a ample and each column represents a protein peak identified by mass. Each cell includes the peak height of the peak for the particular sample. Biomarker Patterns™ Software then performs a classification and regression analysis on the data using parameters selected by the operator. The result of the analysis is a decision tree that includes one or more biomarker peaks that are useful in classification.

The biomarker used in the first split in the tree has strong differentiating characteristics and is subject to further analysis. A sample containing the protein biomaker is subjected to further fractionation to identify a fractionation protocol that produces highly purified biomarker on a ProteinChip® array. The protein is digested with trypsin on chip. The digested protein on the chip is introduced into the probe interface of qQ-TOF mass spectrometer. Several peptide fragments are sequenced. The sequences are submitted to a protein database and a candidate identify is produced with very high confidence of identity.

### Example 2

A genetic or environmental risk factor is known to be associated with a pathophysiological response. Yet, some persons with the risk factor progress and others do not progress as fast or to the same extent if at all. A group of individuals who share the risk factor are identified. The group includes both people who are progressors and nonprogressors. A sample, such as a blood sample or cerebrospinal fluid sample is prepared from each individual. The samples are prepared for profiling by fractionation on a chromatographic column and subsequent analysis as described in Example 1.

### Example 3

An exemplary system for mass spectroscopy data generation and handling is described in this example.

Data generation in mass spectrometry begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOF-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes that are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

### Example 4

Immunoassays can also be used to detect the differentially expressed proteins and biomarkers of the invention. Such assays are useful for screening for modulators of such proteins or biomarkers, as well as for therapeutic and diagnostic applications. Immunoassays can be used to qualitatively or quantitatively analyze such proteins. A general overview of the applicable technology can be found in Harlow & Lane, Antibodies: A Laboratory Manual (1988).

Methods of producing polyclonal and monoclonal antibodies that react specifically with a protein are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science, 246:1275-1281 (1989); Ward et al., Nature, 341:544-546 (1989)).

A number of immunogens comprising portions of differentially expressed protein or biomarker may be used to produce antibodies specifically reactive with the protein or biomarker. For example, recombinant or chemically synthesized polypeptides or an antigenic fragment thereof, can be isolated as described herein. Recombinant protein can be expressed in eukaryotic or prokaryotic cells as described above, and purified as generally described above. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler et al., Eur. J Immunol., 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science, 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 104 or greater are selected and tested for their cross reactivity against non-defensin proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular polypeptide ortholog, such as human polypeptide, can also be made, by subtracting out other cross-reacting orthologs from a species such as a non-human mammal. In this manner, antibodies that bind only to the polypeptide protein may be obtained.

Once the specific antibodies against a protein are available, the protein can be detected by a variety of immunoassay methods. In addition, the antibody can be used therapeutically as a modulator of the protein. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choiceeof). The antibody (e.g., anti-defensin) may be produced by any of a number of means well known to those of skill in the art and as described above.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize defensin protein, or secondary antibodies that recognize anti-defensin.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### Example 5

The methods of the present invention can be applied to the identification of the polypeptide biomarker or factors associated with the progression or nonprogression of a viral disease. For instance, CAF is a protein which is differentially expressed in HIV+ individuals who are relative nonprogressors to AIDS as compared to HIV+ individuals who progressed to AIDS. The protein profiling of these progressor and nonprogressor HIV positive populations can lead to the identification of the factors responsible for the antiviral activity of CAF. See U.S. Patent Application 60/384,428 filed on May 31, 2002 and U.S. Patent Application No. 60/405,595 filed Aug. 23, 2000 and U.S. Patent Application No. 60/412,414 filed Sep. 20, 2003 entitled “Defensins: Use as Antiviral Agents”, assigned to the same assignee as the instant application and herein incorporated by reference in their entireties.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method comprising:
a) profiling a plurality of proteins in a sample from at least one member of a first population exposed to a pathogenic agent wherein the pathogenic agent evokes a pathophysiological response in the first population, whereby the first population is defined as a progressor population;
b) profiling a plurality of proteins in a sample from at least one member of a second population exposed to the pathogenic agent wherein the pathogenic agent does not evoke the pathophysiological response in the second population, whereby the second population is defined as a nonprogressor population; and
c) detecting differentially expressed proteins between the first and second samples.

2. The method of claim 1 wherein the at least one member of a first population is one and the at least one member of a second population is one.

3. The method of claim 1 wherein the pathogenic agent is a pharmaceutical drug or drug candidate and the pathophysiological response is drug toxicity.

4. The method of claim 1 wherein the pathogenic agent is an infectious agent.

5. The method of claim 1 wherein the pathogenic agent is a chemical agent

6. The method of claim 1 wherein the pathogenic agent is a bacterium, a virus or a prion.

7. The method of claim 1 wherein the pathogenic agent is HIV.

8. The method of claim 1 wherein the pathogenic agent is a cancer causing agent.

9. The method of claim 1 wherein the profiling is performed using a method selected from the group consisting of MADLI, SELDI, two-dimensional gel electrophoresis, protein array analysis, population two-hybrid screening, and multiplexed immunoassay.

10. The method of claim 1 further comprising identifying at least one differentially expressed protein.

11. The method of claim 1 wherein detecting comprises detecting a pattern of protein expression that classifies an unknown sample as belonging to the first or second populations.

12. The method of claim 1 wherein the second population is a population immunized against the pathogenic agent.

13. The method of claim 1 wherein the populations are human, non-human animal or plant.

14. A method comprising:
a) docking to a solid support a polypeptide that is differentially expressed between a progressor population and a nonprogressor population;
b) contacting the docked polypeptide with at least one candidate ligand for the protein; and
c) detecting binding between the docked polypeptide and at least one candidate ligand.

15. The method of claim 14 wherein binding is detected by SELDI or immunoassay.

16. A method comprising:
a) docking a plurality of candidate ligands to different addressable locations on at least one solid support;
b) contacting each of the docked candidate ligands with a polypeptide that is differentially expressed between a progressor population and a nonprogressor population; and
c) detecting binding between each of the docked candidate ligands and the polypeptide.

17. The method of claim 16 wherein binding is detected by SELDI or immunoassay.

18. A method comprising:
a) docking one member of a receptor/ligand pair to a solid support, wherein either the receptor or the ligand is a polypeptide that is differentially expressed between progressor and nonprogressor populations;
b) contacting the docked member with the other member of the pair and with a test agent; and
c) determining whether the test agent modulates binding between the receptor/ligand pair.

19. The method of claim 18 wherein binding is detected by SELDI or immunoassay.

20. The method of claim 1, wherein the response differs in severity or time of onset for the first and second populations.

21. The method of claim 1, wherein the at least one member of a first population is at least two and the at least one member of a second population is at least two.

22. The method of claim 1, wherein the plurality of proteins in step a is at least 50 and wherein the plurality of proteins in step b is at least 50.

23. The method of claim 2, wherein the method is repeated for a plurality of samples from each of the first and second populations.
